(19) United States
(12) Patent Application Publication
(10) Pub. No.: US 2019/0105150 A1
(43) Pub. Date: Apr. 11, 2019

(54) SYSTEMS AND METHODS FOR TREATMENT OF AORTIC DISSECTION

(71) Applicant: Aquedeon Medical, Inc., Los Altos, CA (US)

(72) Inventor: Nai-Wen Tsao, Los Altos, CA (US)

(21) Appl. No.: 16/156,466

(22) Filed: Oct. 10, 2018

Related U.S. Application Data
(60) Provisional application No. 62/571,156, filed on Oct. 11, 2017.

(51) Int. Cl. A61F 2/07

(52) U.S. Cl. CPC A61F 2/07; A61F 2002/061; A61F 2240/001

(57) ABSTRACT

The present disclosure involves an ascending aorta graft apparatus and methods for use thereof. The graft is specially designed to treat an aortic dissection or aneurysm occurring at least partially in the region of the ascending aorta and the aortic arch. The ascending aorta graft features at least one bypass or jump graft that establishes fluid communication between two portions of the ascending aorta graft and enables the surgeon to expeditiously reestablish perfusion to at least one artery of the aortic branch immediately upon surgical placement of the ascending aorta graft. An integrated assembly is created with additional stent grafts and a thoracic stent graft that traverses the descending aorta.

### RELATED APPLICATIONS

This application claims priority to and the benefit of U.S. Provisional Patent Application Ser. No. 62/571,156, filed Oct. 11, 2017, which is incorporated in its entirety herein by reference.

### FIELD OF THE PRESENT DISCLOSURE

The present disclosure relates generally to techniques and devices for treatment of aortic dissection or aneurysm with grafts, graft stents and/or related devices to repair a patient's ascending aorta.

### BACKGROUND

An aortic dissection is a serious, life-threatening condition in which the inner layer of the aorta tears or “dissects.” The aorta is the largest blood vessel in the body and establishes critical perfusion between the heart and the vasculature of the rest of the body. Any disease that weakens the strength of the aortic wall, including an aneurysm, may predispose one to aortic dissection. Aortic dissection is defined as the progressive separation of the layers within the aortic wall. Tears in these layers result in the dissection expanding in both directions, followed by blood entering the space in between the layers. As blood enters through the tear, the inner and middle layers of the aorta separate such that inner and outer walls are separated by a blood-filled channel, sometimes called a “false lumen.” When left untreated, about 33% of patients die within the first 24 hours, and 50% die within 48 hours. The 2-week mortality rate approaches 75% in patients with undiagnosed aortic dissection. If the blood-filled channel ruptures through the outer aortic wall, aortic dissection is often fatal without immediate surgical intervention. However, when an aortic dissection is detected early enough for surgical intervention, the chances of survival greatly improve.

Depending on the extent of the anatomic involvement of the dissection, different classifications are applied. In the DeBakey classification, type I aortic dissections originate in the ascending aorta and extend to at least the aortic arch; type II dissections involve the ascending aorta only; and type III dissections begin in the descending aorta, usually just distal to the left subclavian artery and may extend distally away from the aortic arch. In the Stanford classification, type A dissections involve the ascending aorta, while type B dissections are those that do not involve the ascending aorta. Ascending aorta dissections that extend into the aortic arch, i.e. DeBakey type I and Stanford type A with extension to the arch, require emergency surgical repair.

Typically, a dissection of the ascending aorta that extends into the aortic arch or that involves any of the arteries of the aortic arch requires surgical insertion of a graft to replace the diseased portion of the ascending aorta and additional grafts to reestablish blood flow to each artery stemming from the aortic branch where any dissection or disease is present. The procedure may also involve inserting an additional stent that is dedicated to provide blood flow from the ascending aorta to vasculature distal of the descending aorta.

Whenever an open-chest surgical procedure is performed that involves inserting a graft in the ascending aorta and performing any vascular procedure to re-stablish circulation to the arteries of the aortic arch, the surgical procedure is necessarily complex and lengthy. Depending on the extent of disease, the aortic graft devices used, and the surgical techniques employed, a number of technical and clinical considerations can greatly impact the potential for successful repair of the aortic dissection or aneurysm and for eventual recovery of the patient. For example, depending on the procedure, the patient will be placed on cardiopulmonary bypass (CPB) for differing lengths of time. The patient may also be placed on deep hypothermic circulatory arrest (DHCA), a surgical technique that involves cooling the body to temperatures below 20° C. and stopping blood circulation and brain function for up to one hour. DHCA is necessary when blood circulation to the brain must be stopped through the brachiocephalic artery or the left carotid artery of the aortic arch. For obvious reasons, surgical procedures that limit the total amount of time that a patient is placed on CPB or DHCA are preferred.

Similarly, depending on the procedure, fluoroscopy is necessary to image the placement of repair grafts used to re-establish blood flow and it may also be desirable to limit the overall duration of fluoroscopy. Different surgical procedures also have inherently differing potential for excess post-operative bleeding and differing patient recovery times. Closed-chest aortic dissection repair procedures do exist that are performed entirely by endovascular placement of aortic grafts, however, these techniques require extraordinary skill in endovascular techniques and the classification of aortic aneurysms and dissections that may be treated entirely by endovascular techniques is limited. Finally, whether performed by an endovascular or a traditional surgical approach, differing level of surgical technique are required. In some cases, the surgical and endovascular procedures are done on two separate days depending on the nature of the disease and condition of the patient.

Accordingly, a need exists for devices and techniques that improve on the existing devices and techniques for aortic aneurysm and dissection repair. A particular need exists for devices and procedures that reduce the duration of CPB and DHCA, and that optimize other surgical aspects of graft placement to enhance a surgeon's ability to repair the most difficult classifications of aortic aneurysm and dissection and to improve patient outcomes.

### SUMMARY

The present disclosure is directed to a synthetic graft for repairing an aortic dissection. The synthetic graft may include an ascending aortic graft configured to replace a section of a native ascending aorta at a site between a sinotubular junction and a brachiocephalic trunk of a patient, having a body with a first portion and a second portion, wherein the body defines a first fluid communication between the first body portion and the second body portion, at least one jump graft having opposing ends connected to the first body portion and the second body portion and that establishes an additional fluid communication between the first body portion and the second body portion.

In one aspect, the first portion of the body may include an access port establishing an open communication between a proximal opening of the access port and an interior space of the body of the ascending aortic graft.

In one aspect the ascending aortic graft may include an access port for introducing a stent graft and then positioning the stent graft so that a proximal end of the stent graft is within the at least one jump graft and a distal end of the stent graft is within a target artery of the aortic arch.

In one aspect the ascending aortic graft may include an access port for introducing a thoracic stent graft through the access port and positioning a proximal end of the thoracic stent graft within the body of the ascending aorta graft and a distal end of the thoracic stent graft distal of arteries of the aortic arch.

In one aspect, the ascending aortic graft may have three jump grafts.

In one aspect, the at least one jump graft may include a working tube establishing an open connection between a proximal opening of the working tube and an interior space of the jump graft.

In one aspect, the ascending aortic graft further may also have a middle portion disposed between the first portion and the second portion and having a diameter smaller than at least one of a diameter of the first portion and a diameter of the second portion.

In one aspect, the at least one jump graft may have a substantially linear portion along an axial length thereof.

In one aspect, the first fluid communication and each additional fluid communication are independent of each other.

In one aspect, the ascending aortic graft may have one or more markers on the body.

The disclosure also includes a method to repair a damaged aorta. The method may involve replacing a portion of a patient's ascending aorta with an ascending aortic graft at a site between a sinotubular junction and a brachiocephalic trunk, wherein the ascending aortic graft has a body with a first portion proximate to the sinotubular junction and a second portion proximate to the brachiocephalic trunk, wherein the body defines a first fluid communication between the first body portion and the second body portion, and at least one jump graft having opposing ends connected to the first body portion and the second body portion that establishes a first additional fluid communication between the first body portion and the second body portion, sealing proximal and distal ends of the ascending aortic graft at the site to permit blood flow through the ascending aortic graft, establishing blood flow through the ascending aortic graft, the aortic arch, and the descending aorta, at least through the first fluid communication.

In one aspect, a first stent graft may be introduced in the ascending aortic graft and positioned in the at least one jump graft such that a distal end of a first stent graft is advanced into a first target artery of the aortic arch to establish a fluid communication between the ascending aorta and the first target artery through the first additional fluid communication.

In one aspect, a second stent graft may be introduced and positioned such that a distal end of the second stent graft is advanced into a second target artery of the aortic arch to establish a second additional fluid communication between the ascending aorta and the second target artery. A third stent graft may also be introduced and positioned such that a distal end of the third stent graft is advanced into a third target artery of the aortic arch to establish a third additional fluid communication between the ascending aorta and the third target artery.

In one aspect, introducing the first stent graft of the ascending aortic graft may involve introducing the first stent graft in the ascending aortic graft through a proximal opening in a working tube that establishes an open communication to an interior of the at least one jump graft.

In one aspect, the body of the ascending aorta graft may have an access port.

In one aspect, a fluid communication may be established between the ascending aorta and a descending aorta of the patient through the access port by introducing a thoracic stent graft through the access port and positioning a proximal end of the thoracic stent graft within the body of the ascending aorta graft and a distal end of the thoracic stent graft distal of arteries of the aortic arch. The proximal end of the thoracic stent graft may engage an inner intermediate annulus of the ascending aorta graft. The at least one jump graft may span the inner intermediate annulus.

The present disclosure also includes an assembly having three different grafts combined to reestablish perfusion to treat an aortic dissection. The assembly may have:

a) an ascending aortic graft configured to replace a section of a native ascending aorta at a site between a sinotubular junction and a brachiocephalic trunk of a patient, having a body with a first portion and a second portion, wherein the body defines a first fluid communication between the first body portion and the second body portion;

at least one jump graft having opposing ends connected to the first body portion and the second body portion and that establishes an additional fluid communication between the first body portion and the second body portion; and

an access port for introducing a stent graft and then positioning the stent graft so that a proximal end of the stent graft is within the at least one jump graft and a distal end of the stent graft is within a target artery of the aortic arch;

b) at least one stent graft having a length sufficient to establish perfusion between a proximal end of the at least one stent graft when disposed within the at least one jump graft and a distal end when disposed within the target artery wherein the target artery is selected from the group consisting of a brachiocephalic trunk, a left carotid artery, a left subclavian artery, and combinations thereof; and
c) a thoracic stent graft configured to have a proximal end disposed within the body of the ascending aortic graft and a distal end disposed distal of arteries of the aortic arch.

In one aspect, the proximal end of the thoracic stent graft may be configured to engage the ascending aortic graft at an inner intermediate annulus thereof to form a seal thereto, wherein the inner intermediate annulus is positioned in a middle portion of the ascending aortic graft further disposed between the first portion and the second portion and having a diameter smaller than a diameter of the first portion and a diameter of the second portion.

The disclosure also includes a method to assemble an integrated system of at least three different grafts involving placing an ascending aortic graft at a dissected portion of a native ascending aorta, wherein the ascending aortic graft comprises at least one jump graft establishing a fluid communication between a first portion and a second portion, introducing a stent graft through an access port in the jump graft to establish a fluid communication between the at least one jump graft and a target artery of the aortic arch and introducing a thoracic stent graft to position a proximal end thereof within a body of the ascending aortic graft and a distal end thereof distal of arteries of the aortic arch to establish perfusion to vasculature distal of the thoracic stent graft.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 schematically illustrates an ascending aortic graft having been surgically introduced to replace a diseased portion of the ascending aorta, according to an embodiment of the disclosure.

FIG. 2 shows the ascending aorta graft of FIG. 1, with a stent graft introduced to the brachiocephalic artery via one jump graft and its associated working tube, according to an embodiment.

FIGS. 3A and 3B are schematic illustrations of arch-shaped (FIG. 3A) or straight (FIG. 3B) jump graft configurations spanning the body of the aorta stent graft and providing fluid communication between a first and a second portion thereof, according to embodiments of the disclosure.

FIGS. 4A and 4B schematically illustrate an ascending aortic graft, with FIG. 4A showing a middle section of the ascending aortic graft having a reduced diameter compared to the first and second sections and having three jump grafts disposed between the first and second sections of the graft and largely confined within the overall diameter of the first and second portions of the graft as shown in the cross-section along line A-A in FIG. 4B, according to an embodiment of the disclosure.

FIG. 5 is a schematic illustration of an integrated assembly with an ascending aortic graft showing one stent graft disposed between each of the three individual jump grafts and disposed within the brachiocephalic artery, the left carotid artery, and the left subclavian artery, respectively, according to an embodiment of the disclosure.

FIGS. 6A-6D schematically illustrate the assembly of FIG. 5 showing the internal configuration of the ascending aortic graft with the placement of the three stent grafts and the cross-sections of the assembly along lines A-A, B-B, and C-C, respectively according to embodiments of the disclosure.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one having ordinary skill in the art to which the disclosure pertains. For example, the term “suturing” includes drawing two surfaces or edges together with a flexible material to close a puncture, opening, or other wound, wherein the suture is a material that may be synthetic or natural, such as a polymer, gut, metallic wire or other suitable equivalents.

Finally, as used in this specification and the appended claims, the singular forms “a, “an” and “the” include plural referents unless the content clearly dictates otherwise.

All of the blood delivered from the heart to the remaining tissues of the body passes through the aorta. The ascending aorta begins at the opening of the aortic valve at the sinotubular junction above the left ventricle of the heart and is connected to the aortic arch that curves above the heart between the ascending and descending aorta. Three major arteries branch off from the superior arterial wall of the aortic arch to supply blood to the tissues of the superior regions of the body: the brachiocephalic trunk (or innominate artery) that branches into the right carotid and right subclavian arteries, the left common carotid artery, and the left subclavian artery. The brachiocephalic trunk is the first or most proximate artery to arise from the aortic arch, carrying blood to the right arm and the right side of the head and neck. The left common carotid artery in the middle supplies blood to the left side of the head and neck. The most distal artery arising from the aortic arch is the left subclavian artery and supplies blood to the left arm.

The term “aortic dissection” as used herein encompasses a spectrum of life-threatening aortic conditions potentially with pathologic overlap, and with differing severity of characteristic symptoms, but all having substantial risk in common in the absence of critical surgical intervention. Acute dissection includes the presence of aortic hematoma, aneurysms, penetrating ulcers, and implies the formation of an intimal tear in the layers of the aorta with progression to separation between the tissue layers and a likelihood of subsequent anterograde blood flow into a false lumen between the tissue layers. Localized bleeding or entry of blood into the pericardium may be present.

The DeBakey and Stanford classification systems may be correlated to disease extent. The grafts according to this disclosure are configured for specific use in procedures to alleviate aortic dissection in the Stanford classification, type A aortic dissections involving the ascending aorta (regardless of the site of the primary intimal tear) and extending beyond the ascending aorta to any extent in the aortic arch and associated arteries, but not to the exclusion of type A/B dissections that include all other anatomic aortic locations beyond the ascending aorta. In the DeBakey classification scheme, the devices and techniques of the disclosure are applicable to type I dissections involving the ascending aorta with distal extension, but not to Type II aneurysms and dissections that are entirely limited to the ascending aorta alone or type III aneurysms and dissections that are limited to the descending aorta.

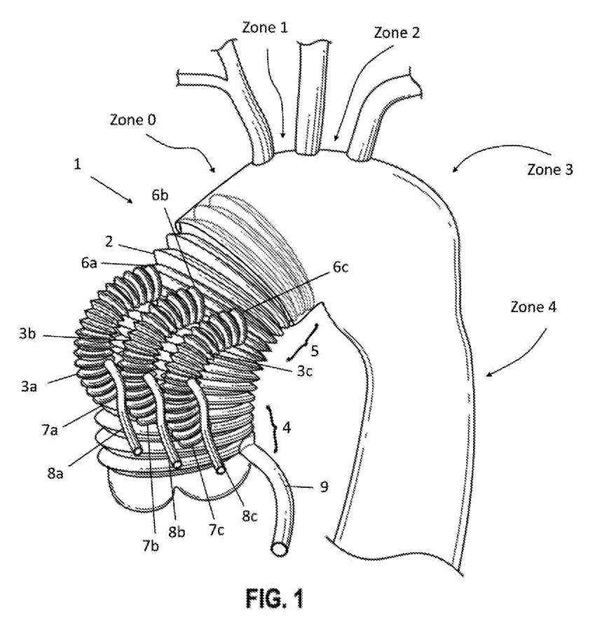

Different landing zones are defined for reference when describing the regions of the aorta and for the purpose of orienting the placement and positioning of the ascending aortic graft, the stent grafts, and the thoracic stent graft. Zone 0 is in the ascending aorta, proximal to the brachiocephalic trunk. Zone 1 covers the portion of the arch between the brachiocephalic trunk and the left common carotid artery. Zone 2 covers the part of the aortic arch between the left common carotid artery and the left subclavian artery. Zone 3 covers the proximal descending thoracic aorta distal to the left subclavian artery. Zone 4 covers the descending aorta. These zones are indicated in FIG. 1.

The present disclosure involves an ascending aorta graft specially designed to enhance a surgeon's ability to treat an aortic dissection, or aneurysms occurring at least partially in the region of the ascending aorta and at least one of the arteries of the aortic arch. The ascending aorta graft features at least one bypass or jump grafts that establishes fluid communication between two portions of the body of the ascending aorta graft and enables the surgeon to expeditiously reestablish perfusion to at least one artery of the aortic branch immediately upon surgical placement of the ascending aorta graft. One distinct advantage of the graft is that the surgeon can repair a damaged portion of the native ascending aorta by placement of the ascending aortic graft of the disclosure and, without additional surgical interventions, and reestablish normal perfusion to the brain and heart. Subsequently, through at least one access port disposed in the body of the ascending aorta graft, the surgeon can place additional stent grafts to target arteries.

Accordingly, the surgeon may replace a portion of the ascending aorta with the ascending aorta graft of the disclosure as a single step under CPB, reestablish normal perfusion, and then subsequently use conventional stent grafts introduced through the jump grafts or the body of the ascending aortic graft, to establish reperfusion to at least one artery of the aortic branch. Because of the design of the graft of the disclosure, novel procedures are enabled wherein the stent grafts that establish perfusion one or more arteries of the aortic branch is achieved while the heart is beating normally, thereby limiting the length of time of CPB and eliminating DHCA.

Furthermore, the procedures enabled by the novel design of the disclosure allow assembly of an integrated system of at least three different grafts, wherein the assembly occurs entirely through the graft placed in the descending aorta or Zone 0.

The ascending aorta graft is designed to have at least one bypass or jump graft for each artery of the aortic arch that will be targeted for reestablishment of perfusion by placing a stent graft. In a preferred embodiment, the ascending aorta graft of the disclosure has three bypass or jump grafts that span between two regions of the body of the graft to establish perfusion between a first portion (proximal) portion and a second (distal) portion of the body of the ascending aorta graft. The body of the ascending aorta graft optionally has at least one access port with an opening at a proximal end thereof and a distal end traversing the body of the graft to permit open communication with the fluid path within the internal portion of the body of the ascending aorta graft.

In a preferred embodiment, each jump graft has a working tube disposed therein having an opening at a proximal end and a distal end traversing the body of the jump graft to permit open communication with the fluid path within the internal portion of the jump graft. Because the proximal portion of the jump graft is connected to the first portion of the ascending aorta graft, perfusion may be established between the ascending aorta and a target artery by passing a stent graft through the jump graft that is accessed by passing the stent graft through the working tube. After introducing the stent graft through the working tube of the jump graft, the stent graft is advanced through the interior of the jump graft, through the interior of the second portion of the body of ascending aorta graft and directed end advanced to the target artery until the distal end of the stent graft is located in a desired position within the target artery.

By this technique, placement of the ascending aorta graft comprising the at least one jump graft, enables the establishment of perfusion between the first or proximal portion of the ascending aorta graft, via the stent graft introduced through the jump graft, to the target artery. As noted, this technique can be performed entirely while the heart is beating normally, i.e. following termination of CPB and for any number of target arteries within the aortic branch.

Depending on the physiology of the aortic dissection or aneurysms, an additional thoracic stent graft may be introduced by any of several techniques. The thoracic stent traverses the descending aorta and the aortic arch to create fluid communication between the ascending aortic graft and the remaining vasculature of the body distal of the thoracic stent graft. The thoracic stent graft may be introduced by an endovascular technique concurrently with, or following, the placement of the ascending aorta graft. The thoracic stent graft may also be introduced through the dedicated access port disposed in the body of the ascending aorta graft. In one embodiment, the proximal portion of the thoracic stent graft is sized and shaped to be placed in close, mating engagement with a matching structure disposed in the interior of the body of the ascending aorta graft.

The body of the ascending aorta graft and the shape of the bypass or jump grafts may be designed in any of several configurations that provide the ability for fluid communication between a first and second portion of the body of the ascending aorta graft. The jump grafts may be arched, straight, or disposed to lie parallel to a separate middle section of the body of the ascending aorta graft, located between the first (proximal) and second (distal) portions of the graft. In some embodiments, the grafts can be made of polyester, Dacron or PTFE

By using the ascending aorta graft of the disclosure, a number of novel surgical procedures are enabled. First, as noted above, the surgeon may establish perfusion between the proximal portion of the ascending aorta graft and any one or more of the arteries of the aortic branch without additional surgical intervention to any of the body of the native ascending aorta, the body of the native aortic arch, the native lumen of any of the aortic branch arteries, or the native descending aorta. Second, the surgeon may use a common access point, in this case, the surgically inserted ascending aorta graft, to introduce stent grafts to any target artery by passing the stent graft through the internal space of the body of the ascending aortic graft and advancing each stent graft to the target artery. Third, again, using only the ascending aorta graft, the surgeon may introduce at least one stent graft and a thoracic stent graft after CPB has been discontinued, without DHCA, and absent additional surgical intervention. Those of ordinary skill in the art will appreciate any number of additional novel surgical techniques and discrete procedures enabled by the unique structure of the ascending aorta graft of the present disclosure to reduce total surgical procedure times and improve clinical outcomes.

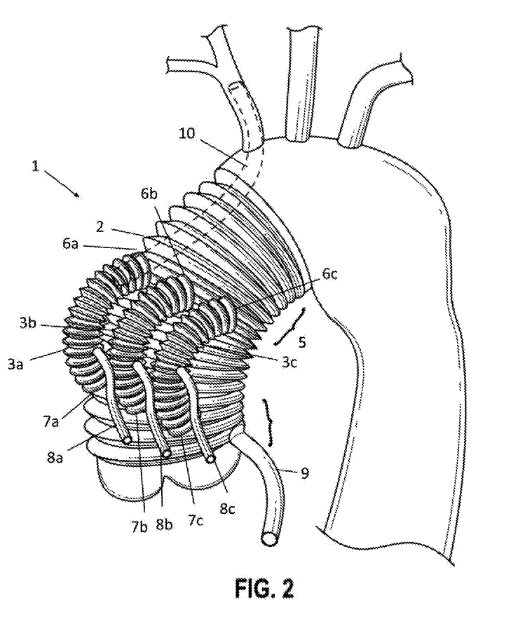

Referring to FIG. 1, the ascending aorta graft 1 of the present disclosure has been surgically introduced to replace a diseased portion of the ascending aorta. The embodiment of FIG. 1 shows the first and second portions of the ascending aortic graft, arch-shaped bypass or jump grafts, a working tube associated with each jump graft and a dedicated access port traversing the body of the graft. As such, the aorta stent graft 1 is comprised of a body 2 having a first portion 4 attached to the proximal portion of the ascending aorta and proximate to the sinotubular junction. The body 2 of the graft 1 is further comprised of a second portion 5 attached to the most distal portion of the ascending aorta and proximate to the brachiocephalic trunk, with the distal end of the second portion 5 in phantom within the aorta. In the FIGS. 2, 5 and 6A, the most distal portion of the ascending aorta is omitted for clarity. The body 2 of the graft 1 is comprised of at least one bypass or jump graft. In the embodiment of FIG. 1, three jump grafts 3a, 3b, 3c are shown although any structure that performs the function of establishing a fluid communication between the first portion 4 and the second portion 5 may be used. Accordingly, body 2 of graft 1 establishes a first fluid communication between the first portion 4 of the body 2 and the second portion 5 of the body 2, while each jump graft 3 establishes an additional fluid communication between opposing ends of the jump graft 3, which are disposed at the first portion 4 and the second portion 5 of the body 2 of graft 1.

Generally, the first fluid communication is made through the interior of body 2 of graft 1 and additional fluid communications are made through the interior of each jump graft 3, such as a second fluid communication through jump graft 3a, a third fluid communication through jump graft 3b, and/or a fourth fluid communication through jump graft 3c. The first fluid communication and the additional fluid communications may be substantially independent, in that fluid flow through the first fluid communication is substantially unaffected by conditions within the additional fluid communications and fluid flow through each additional fluid communication is substantially unaffected by conditions within the first fluid communication and the other additional fluid communications. As will be appreciated, this facilitates performing operations within the jump grafts 3a, 3b, 3c, such as to establish stent grafts to perfuse arteries served by the aortic arch as discussed below, in a manner that does not perturb fluid flow through body 2 of graft 1, improving perfusion to the associated anatomy, including by maintaining flow to the descending aorta. Likewise, operations within each jump graft may be performed without affecting fluid flow in the other jump grafts.

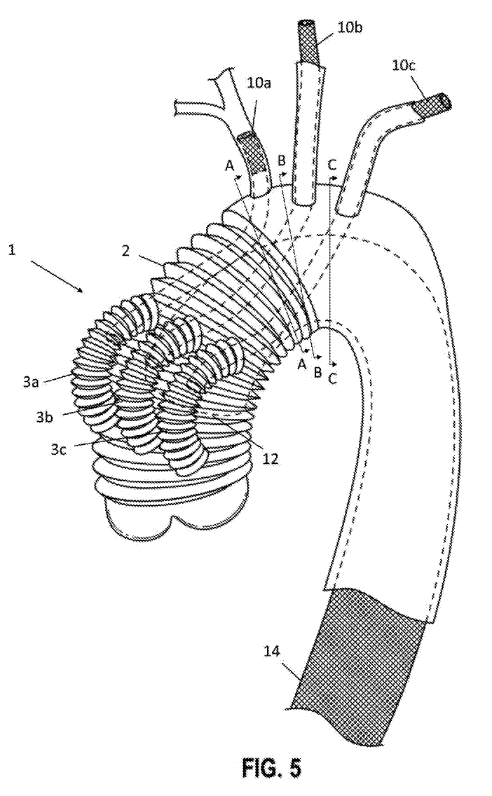

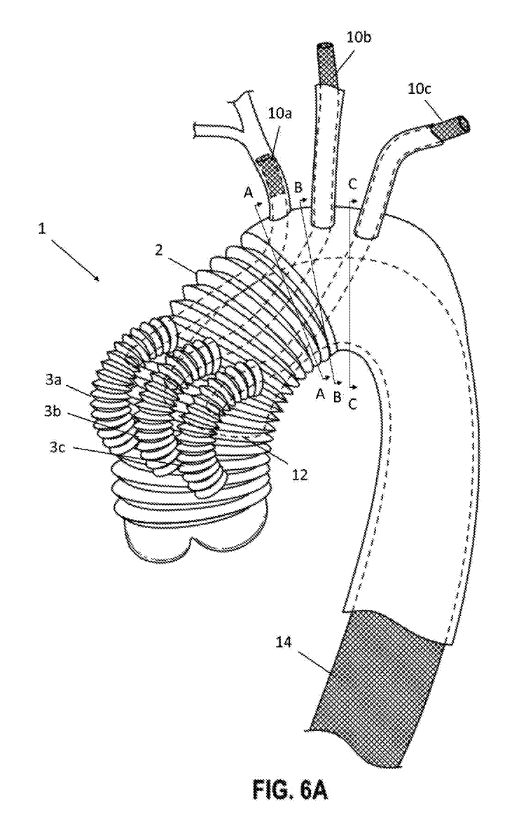

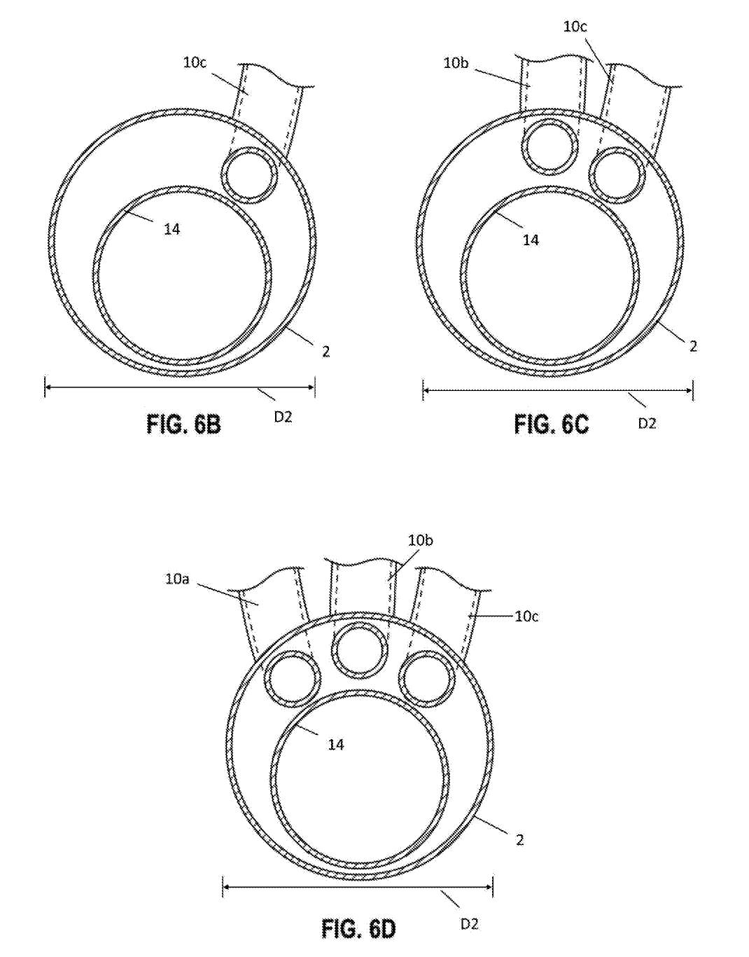

Establishing a fluid communication between the first portion 4 and the second portion 5 establishes a point wherein additional stent grafts can be introduced to provide perfusion to any of the arteries of the aortic arch, such as through an access port. In the embodiment of FIG. 1, each jump graft 3a, 3b and 3c are each comprised of a single working tube 8a, 8b, 8c providing a passage to the interior of each jump graft 3a, 3b, 3c. The body 2 of the graft 1 is further comprised of an access port 9 traversing the body of the graft 1 and also providing access to the interior of the body 2. The access port 9 is preferably disposed in the first portion 4 and may be used as an alternative to the working tubes 8a, 8b, 8c for the introduction of stent grafts between any of jump graft 3a, 3b, 3c and a target artery or may be used to introduce a thoracic stent graft (not shown, see FIGS. 5 and 6).

In the embodiment of FIG. 1, the jump grafts 3a, 3b, 3c are shown in an arched configuration that spans the first, proximal portion 4 and the second, distal portion 5 of the body to establish a fluid communication therebetween. Accordingly, each of the jump grafts 3a, 3b, 3c, has a proximal opening 7a, 7b, 7c such that each proximal end of the individual jump graft is attached about its periphery to a conforming opening in the first portion 4 of the body 2. In a similar fashion, each of the jump grafts 3a, 3b, 3c has a distal opening 6a, 6b, 6c such that the distal end of the individual jump graft 3a, 3b, 3c is attached about its periphery to a conforming opening in the second portion 5 of the body 2.

Each jump graft 3a, 3b, 3c provides a landing zone in Zone 0 for operative, fluid communication to an artery of the aortic arch to establish perfusion from the ascending aorta to the target artery. The length of the landing zone is preferably greater than 2 cm to provide spacing and an overall length for each jump graft 3a, 3b, 3c such that the stent graft (not shown, see FIG. 2) offers sufficient engagement between the stent graft 10 and each jump graft 3a, 3b, 3c.

Referring to FIG. 2, one of ordinary skill in the art will appreciate that the size of the individual structures of the present disclosure as shown in the figures is only representative and that the overall diameters of each of the graft 1, the body portions 2, 4, 5, as well as the jump graft 3, and the access port 9 or the length of the body 2 both proximal and distal to the attachment points of the jump graft 3 may vary both by manufacturing and design choices as well as the underlying physiology of the patient. Accordingly, ascending aortic grafts 1 may be sized in discrete lengths or diameters for the surgeon to select depending on the patient's particular anatomy and specifically the length and diameter of an individual patient's ascending aorta.

As shown in FIG. 2, a stent graft 10 is an elongated lumen that provides fluid communication between the interior space of a jump graft 3 to a target artery of the aortic arch. The size of the working tube 8 may also be sized accordingly to accommodate introduction of the stent graft 10 through the working tube 8. The stent graft 10 has a proximal end and a distal end such that the proximal end is disposed in the body of the jump graft 3 and the length of the stent graft 10 traverses the ascending aortic graft 1, a portion of the aortic arch and has a distal end disposed in the target artery. Preferably, the distal end of the stent graft 10 is advanced a sufficient distance within the target artery to provide adequate perfusion from the jump graft 3 through the stent graft 10. The length of the stent graft 10 is held in place by frictional engagement along its length and at the proximal and distal ends such that a permanent fluid communication path is established for perfusion of the target artery. The frictional engagement may be achieved through a self-expanding or balloon-expandable feature in the stent graft. The stent graft 10 may be deployed using standard endovascular/interventional methods, using catheters and guidewires, under x-ray fluoroscopy guidance.

As noted above, in this configuration, the surgeon can establish the potential for creating new perfusion pathways to each of the arteries of the aortic arch as well as to the entire vasculature of the patient distal to the aortic arch and the descending aorta solely by manipulation of the ascending aortic graft 1 to introduce additional grafts through the ascending aorta. Additional perfusion-creating grafts, such as one or more of the stent grafts 10 as well as a companion thoracic stent graft (not shown, see FIGS. 5 and 6) are placed without additional CPB, DHCA or other surgical intervention of the target arteries. Accordingly, techniques of this disclosure involve placement of an ascending aortic graft in Zone 0 and establishing perfusion distal to Zone 0 including to the arteries of the aortic arch, the descending aorta, and vasculature distal each or any of the ascending aorta, the aortic arch, or the descending aorta without additional surgical intervention at the aortic branch arteries or the remaining anatomical structures of the descending aorta, including placement of the thoracic stent graft through either of the access port 9 or through an endovascular technique as part of a novel hybrid approach. Moreover, the entire treatment may be completed in a single procedure without the need for aortic arch repair on one day and thoracic stent graft placement on a different day.

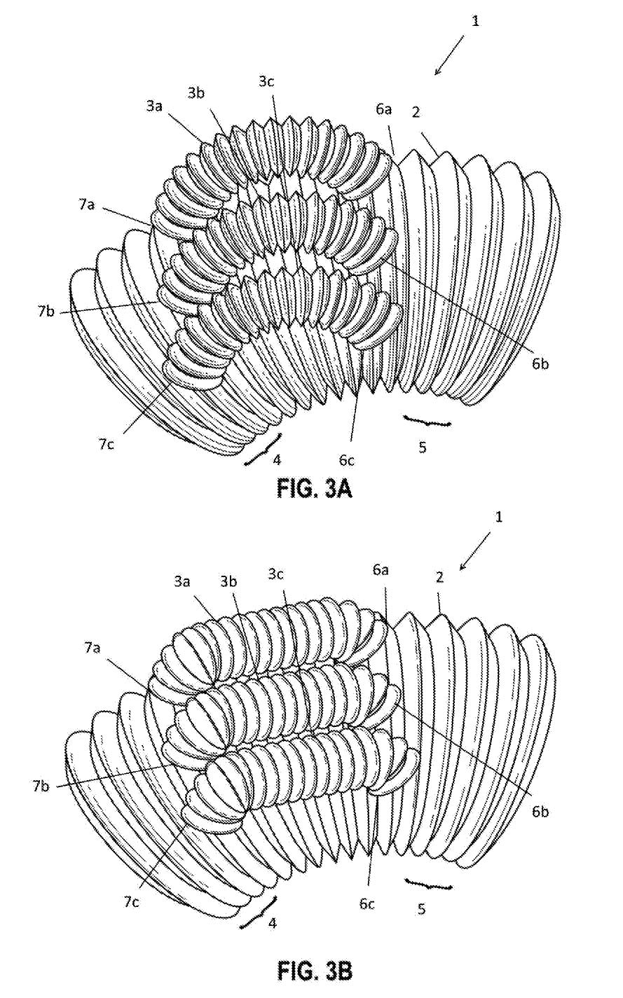

Referring to FIGS. 3A and 3B, the bypass or jump grafts of the present disclosure may have arch-shaped (3A) or straight (3B) configurations of the jump graft 3a, 3b, 3c spanning the body 2 of the aorta graft 1 and, similar to the embodiments of FIGS. 1 and 2, establish fluid communication between a first and a second portion 4, 5 of the body 2 of the graft 1 and similarly provide an access port or other point for introducing the stent grafts (not shown) for re-establishing perfusion in the target arteries. FIGS. 3A and 3B are illustrated the potential for a number of different possible configurations for the jump grafts 3a, 3b, and 3c. Additionally, radiopaque markers may be incorporated into the ascending aortic graft 1 near the anastomoses sites 6a, 6b and 6c to facilitate the placement of stent grafts 10a, 10b and 10c connecting the ascending graft 1 to the branch vessels, under fluoroscopy.

Accordingly, the other structures such as the working tubes and access port as described in FIGS. 1 and 2 can readily be provided in the embodiments of FIGS. 3A and 3B. Furthermore, as noted above, the embodiment of FIGS. 3A and 3B illustrate three jump grafts 3a, 3b 3c, but the ascending aortic graft 1 of the present disclosure is comprised of at least one jump graft 3, and differing embodiments of the graft 1 can be manufactured that feature differing numbers of jump grafts 3, including embodiments that match the number of jump grafts 3 to the number of target arteries of the aortic arch where stent grafts are to be introduced. Accordingly, for a patient with an aortic dissection affecting one, two, or three target arteries of the aortic branch, the surgeon may select an embodiment of the ascending aortic graft 1 of the present disclosure having one, two, or three jump grafts 3 as is dictated by the anatomical status of the patient and the extent of the aortic dissection.

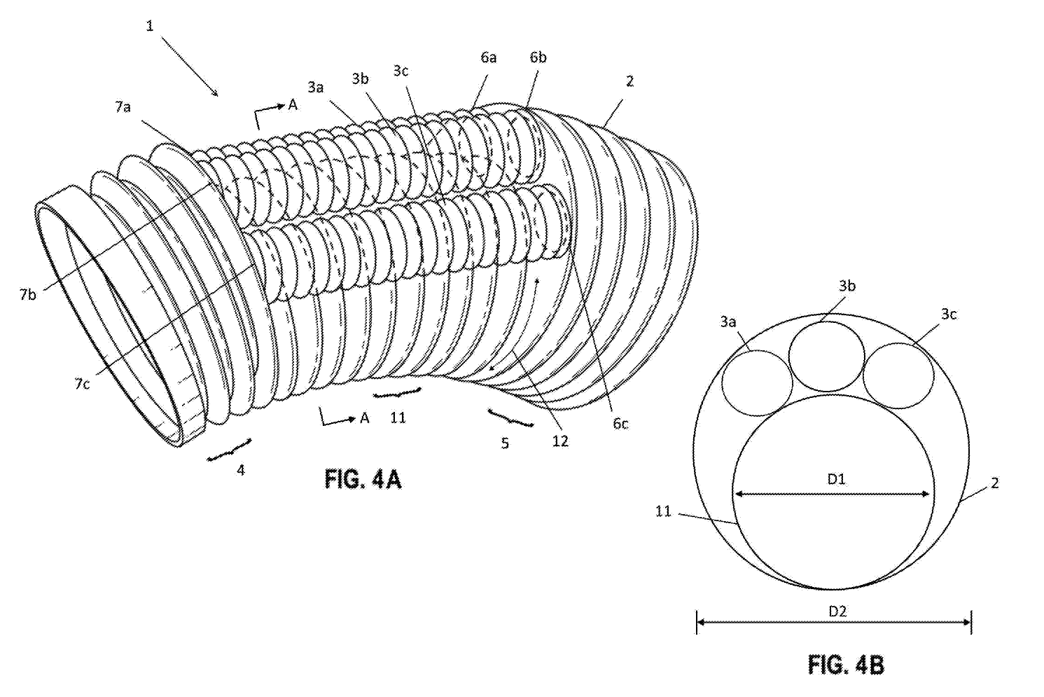

Referring to FIGS. 4A and 4B, an embodiment of the ascending aortic graft 1 has a first and second sections and having three jump grafts 3a, 3b, 3c disposed between the first and second sections 4,5 of the graft 1 and largely confined within the overall diameter D2 of the first and second portions 4,5 of the graft 1. An internal portion of the graft 1 may comprise an annulus 12 between the second section 5 and the middle section 11 may be sized and configured to engage the proximal portion of a thoracic stent graft (not shown, see FIGS. 5 and 6).

In the embodiment of FIGS. 4A and 4B, the first, proximal portion 4 has the diameter D2 and, as in the previous figures, each of the jump grafts 3a, 3b, 3c provide fluid communication between the first and second portions 4, 5. The middle section 11 has a reduced diameter D1 that may permit the sum of the individual diameters of each of the jump grafts 3a, 3b, 3c added to the diameter D1 of the middle portion 11 to be no greater than the diameter D2 of the first and second portions 4, 5 as schematically indicated in the cross-sectional view of FIG. 4B, taken along line A-A in FIG. 4A. In the present embodiment, the diameter D1 may be in a range from 15 mm to 25 mm Suitably, the diameter D1 of middle portion 11 is 80% of diameter D2 of the first and second portions 4, 5. The diameter of each of jump grafts can be in a range of 6 mm to 12 mm. The diameter D2 of the first and second portions 4, 5 can be chosen to be sized in a variety of diameters of aorta. In addition, the diameter D2 of the first and second portions 4, 5 is chosen to be able to sustain the blood pressure, for example, 140 mmHg. In other embodiments, diameter D1 may be no greater than at least one of the diameters of first and second portions 4, 5.

As with the embodiments of FIGS. 3A and 3B the other structures such as the working tubes and access port as described in FIGS. 1 and 2 can readily be provided in the embodiment of FIGS. 4A and 4B and any number of jump grafts 3 can be provided in the configuration and orientation of FIGS. 4A and 4B including manufacturing individual embodiments featuring differing numbers of jump grafts 3 to match the number of jump grafts 3 to the number of target arteries of the aortic arch where stent grafts are to be introduced.

Other embodiments of FIGS. 3A and 3B may be feasible. For example the proximal anastomoses sites of the jump grafts 7a, 7b and 7c could be combined into a single and larger anastomosis with a single access port. The single access port may be used to deploy one or more stent grafts to one or more branch vessels of the aortic arch.

Referring to FIG. 5, an integrated assembly unique to the present disclosure provides a complete solution to an aortic dissection affecting the ascending aorta and at least one artery of the aortic arch that is deployed entirely from Zone 0. The assembly is comprised of the ascending aortic graft 1 as described herein and includes additional stent grafts 10a, 10b, 10c and a thoracic stent graft 14. In this embodiment, thoracic stent graft 14 also traverses the descending aorta and the aortic arch to a position within the interior of the body of the ascending aortic graft to establish fluid communication between the ascending aorta and the remaining vasculature of the body distal to the thoracic stent graft. For example, the distal end of thoracic stent graft 14 may extend distally of the arteries of the aortic arch, such as the most distal artery, the left subclavian artery. As noted previously, the only requirement for creating an assembly according to this disclosure is the deployment of at least one stent graft 10 through at least one jump graft 3. The illustration of three jump grafts 3a, 3b, 3c, and three stent grafts 10a, 10b, 10c is exemplary only and any number matching target arteries is contemplated. Moreover, the integrated assembly may include stent graft 10 and a thoracic stent graft 14 that are off-the-shelf or generic and may not be specifically designed for use with the ascending aortic graft 1 of the disclosure. Accordingly, the use of the ascending aorta graft 1 of the disclosure includes generating an assembly of otherwise off-the-shelf components to yield a complete perfusion solution for an aortic dissection that otherwise would have to be treated with a variety of specialized devices.

Moreover, the entire deployment of the integrated assembly can be achieved entirely from Zone 0 and via the working tubes 8 or the access port 9 of the ascending aortic graft 1 (as shown in FIG. 2). In this embodiment, referring to FIGS. 5 and 6A, the thoracic stent graft 14 also traverses the descending aorta and the aortic arch to a position within the interior of the body 2 of the ascending aortic graft 1 to establish fluid communication between the ascending aorta and the remaining vasculature of the body distal to the thoracic stent graft 14. As noted above, with respect to the thoracic stent graft 14, the distal end thereof may engage an annulus 12 that is interior to the body 2 of the graft 1 to provide a sealing engagement between the thoracic stent graft 14 and the interior annulus 12 such that a sealed perfusion pathway is provided from Zone 0 through the ascending aorta graft 1, through the aortic arch, and to the distal vasculature of the patient. Although, as noted above, the stent grafts 10 and the thoracic stent graft 14 may be off-the-shelf components, the proximal end of the thoracic stent graft 14 may also have a tapered end (not shown) meant to engage the inner annulus 12 of the ascending aorta graft 1 to provide a meeting engagement within the body of the graph 1 and to provide additional space and clearance for passage of the stent grafts 10 exiting from the jump grafts 3 along the length of the distal end of the thoracic stent graft 14 through the portion of the aortic arch to reach the target artery(ies).

Referring to FIGS. 6A-6D, the assembly of FIGS. 5 and 6A has an internal configuration of the ascending aortic graft 1 with the placement of the three stent grafts 10a, 10b, 10c. Cross-sections of the assembly are shown along lines A-A, B-B, and C-C, respectively showing the disposition and orientation of the thoracic stent graft 14 disposed within the diameter of the body 2 of the aortic stent graft 1. Referring to FIG. 6A, an exemplary embodiment of the present disclosure is in assembly where three stent grafts 10a, 10b, 10c are introduced through working tubes 8a, 8b, 8c (not shown, see FIGS. 1 and 2) to engage each of the brachiocephalic trunk, the left carotid artery, and the left subclavian artery, respectively. As shown in the cross-section represented in FIG. 6B, a long line C-C, the thoracic stent graft 14 and the distal end of the stent graft 10c dedicated to the left subclavian as the target artery are disposed within the outer diameter D2 of the body 2 of the ascending aorta graft 1.

Similarly, in the embodiment of FIG. 6C, along the cross-section represented by line B-B, the thoracic stent graft 14 and the distal end of the two stent grafts 10b, 10c dedicated to the left subclavian and left carotid arteries, respectively, are disposed within the outer diameter D2 of the body 2 of the ascending aortic graft 1. In the embodiment of FIG. 6 D, along the cross-section represented by line A-A, the thoracic stent graft 14 and the distal end of the three stent graft's 10a, 10b, 10c dedicated to the brachiocephalic artery, the left subclavian, and the left carotid arteries, respectively are disposed within the outer diameter D2 of the body 2 of the ascending aortic graft 1.

As noted above, the design of grafts according to this disclosure enables the performance of unique surgical procedures for the repair of an aortic dissection, or aneurysm including the procedure of combining separate grafts to create an integrated assembly to reestablish perfusion from the ascending aorta to at least one target artery of the aortic arch and the vasculature distal of the descending aorta.

Described herein are certain exemplary embodiments. However, one skilled in the art that pertains to the present embodiments will understand that the principles of this disclosure can be extended easily with appropriate modifications to other applications.

What is claimed is:

1. A synthetic graft for repairing an aortic dissection or aneurysm comprising:
an ascending aortic graft configured to replace a section of a native ascending aorta at a site between a sinotubular junction and a brachiocephalic trunk of a patient, having a body with a first portion and a second portion, wherein the body defines a first fluid communication between the first body portion and the second body portion; and
at least one jump graft having opposing ends connected to the first body portion and the second body portion and that establishes an additional fluid communication between the first portion and the second portion.

2. The synthetic graft of claim 1, wherein the ascending aortic graft comprises three jump grafts.

3. The synthetic graft of claim 1, wherein the at least one jump graft comprises a working tube establishing an open connection between a proximal opening of the working tube and an interior space of the jump graft.

4. The synthetic graft of claim 1, wherein the first portion of the body comprises an access port establishing an open communication between a proximal opening of the access port and an interior space of the body of the ascending aortic graft.

5. The synthetic graft of claim 1, wherein the ascending aortic graft further comprises a middle portion disposed between the first portion and the second portion and having a diameter smaller than at least one of a diameter of the first portion and a diameter of the second portion.

6. The synthetic graft of claim 1, wherein the at least one jump graft has a substantially linear portion along an axial length thereof.

7. The synthetic graft of claim 1, wherein the first fluid communication and each additional fluid communication are independent of each other.

8. The synthetic graft of claim 1, wherein the ascending aortic graft further comprises at least one markers on the body.

9. The synthetic graft of claim 1, further comprising at least one access port.

10. A method to repair a damaged aorta comprising:
replacing a portion of a patient's ascending aorta with an ascending aortic graft at a site between a sinotubular junction and a brachiocephalic trunk, wherein the ascending aortic graft has a body with a first portion proximate to the sinotubular junction and a second portion proximate to the brachiocephalic trunk, wherein the body defines a first fluid communication between the first portion and the second portion, and at least one jump graft having opposing ends connected to the first portion and the second portion that establishes a first additional fluid communication between the first portion and the second portion;
sealing proximal and distal ends of the ascending aortic graft at the site to permit blood flow through the ascending aortic graft; and
establishing blood flow through the ascending aortic graft, the aortic arch, and the descending aorta, at least through the first additional fluid communication.

11. The method of claim 10, wherein the body of the ascending aorta graft further comprises an access port.

12. The method of claim of claim 11, further comprising introducing a first stent graft in the ascending aortic graft through the access port; and
positioning the at least one jump graft such that a distal end of the first stent graft is advanced into a first target artery of the aortic arch to establish a fluid communication between the ascending aorta and the first target artery through the first additional fluid communication.

13. The method of claim 12, further comprising introducing a second stent graft and positioning the second stent graft such that a distal end of the second stent graft is advanced into a second target artery of the aortic arch to establish a second additional fluid communication between the ascending aorta and the second target artery.

14. The method of claim 13, further comprising introducing a third stent graft and positioning the third stent graft such that a distal end of the third stent graft is advanced into a third target artery of the aortic arch to establish a third additional fluid communication between the ascending aorta and the third target artery.

15. The method of claim 12, wherein introducing the first stent graft in the ascending aortic graft through the access port comprises introducing the first stent graft in the ascending aortic graft through a proximal opening in a working tube that establishes an open communication to an interior of the at least one jump graft.

16. The method of claim 11, further comprising establishing a fluid communication between the ascending aorta and a descending aorta of the patient through the access port by introducing a thoracic stent graft through the access port and positioning a proximal end of the thoracic stent graft within the body of the ascending aorta graft and a distal end of the thoracic stent graft distal of arteries of the aortic arch.

17. The method of claim 16, wherein the proximal end of the thoracic stent graft engages an inner intermediate annulus of the ascending aorta graft.

18. The method of claim 17, wherein the at least one jump graft spans the inner intermediate annulus.

19. An assembly having three different grafts combined to reestablish perfusion to treat an aortic dissection or aneurysm comprising:
a) an ascending aortic graft configured to replace a section of a native ascending aorta at a site between a sinotubular junction and a brachiocephalic trunk of a patient, having a body with a first portion and a second portion, wherein the body defines a first fluid communication between the first portion and the second portion;
at least one jump graft having opposing ends connected to the first portion and the second portion and that establishes an additional fluid communication between the first portion and the second portion; and
an access port for introducing a stent graft and then positioning the stent graft so that a proximal end of the stent graft is within the at least one jump graft and a distal end of the stent graft is within a target artery of the aortic arch;
b) at least one stent graft having a length sufficient to establish perfusion between a proximal end of the at least one stent graft when disposed within the at least one jump graft and a distal end when disposed within the target artery wherein the target artery is selected from the group consisting of a brachiocephalic trunk, a left carotid artery, a left subclavian artery, and combinations thereof; and
c) a thoracic stent graft configured to have a proximal end disposed within the body of the ascending aortic graft and a distal end disposed distal of arteries of the aortic arch.

20. The assembly of claim 19, wherein the proximal end of the thoracic stent graft is configured to engage the ascending aortic graft at an inner intermediate annulus thereof to form a seal thereto and wherein the inner intermediate annulus is positioned in a middle portion of the ascending aortic graft further disposed between the first portion and the second portion and having a diameter smaller than a diameter of the first portion and a diameter of the second portion.

21. A method to assemble an integrated system of at least three different grafts comprising:
placing an ascending aortic graft at a dissected or aneurysmal portion of a native ascending aorta, wherein the ascending aortic graft comprises at least one jump graft establishing a fluid communication between a first portion and a second portion;
introducing a stent graft through an access port in the jump graft to establish a fluid communication between the at least one jump graft and a target artery of the aortic arch; and
introducing a thoracic stent graft to position a proximal end thereof within a body of the ascending aortic graft and a distal end thereof distal of arteries of the aortic arch to establish perfusion to vasculature distal of the thoracic stent graft.
